(19) United States
(12) Patent Application Publication
(10) Pub. No.: US 2011/0104670 A1
(43) Pub. Date: May 5, 2011

(54) METHOD, DEVICE AND MOLECULAR BIOLOGY KIT FOR EXTRACTING AMPLIFIED GENETIC MATERIAL

(75) Inventor: Yvon Cayre, Paris (FR)

(73) Assignee: METAGENEX, Paris (FR)

(21) Appl. No.: 12/679,027

(22) PCT Filed: Sep. 15, 2008
(86) PCT No.: PCT/FR2008/051650

§ 371 Date: Mar. 19, 2010

(30) Foreign Application Priority Data
Sep. 21, 2007 (FR) 0757779
Sep. 1, 2008 (FR) 0855849

(51) Int. Cl. C12Q 1/68; C12N 5/00; C12P 19/34; C12M 1/33

(52) U.S. Cl. 435/6; 435/325; 435/91.2; 435/306.1

(57) ABSTRACT

A method for collecting cellular material from particular cells present in a liquid, that includes: a step (210) of inserting the liquid into a compartment (105) through an upper opening (106) in the compartment, the compartment having a lower opening (107) while between the openings is provided a filter (115) in which the micropores have a diameter between that of the particular cells and that of other cells; a filtration step (215) during which the major portion of the liquid and the other cells flows through the filter; a step (230) of DNA and/or RNA lysing and amplifying; and a step (250) of recovering on the filter the amplified genetic material.

The present invention concerns a method, a device and a molecular biology kit for extracting amplified genetic material from isolated cells on filters and for detecting mutations and levels of expression of genes coding for sensitivity and resistance to the targeted therapies.

It applies in particular to collecting and uniformly amplifying DNA or RNA from particular cells present in a liquid, notably blood.

Some particular cells of the blood, for example tumor cells or trophoblastic cells, are present in very low concentrations and must be concentrated for cytopathologic analysis. However, compared to the blood cells, they are of larger size.

It is known, for example from the document PCT/FR 2006/000562, to apply a formaldehyde-based fixing buffer to a blood sample to fix the cells searched for, and then to pass the resulting liquid through a porous filter. That filter is then analyzed in the laboratory to search for the cells therein under a microscope. They can thereafter be sampled on the filter for analysis, for example by genetic analysis. However, this procedure cannot be reproduced on a large scale at reasonable cost because of the time, the equipment and the precise work that it entails.

This would enable molecular biological analyses to be carried out at the same time on tumor cells and on trophoblastic cells.

The present invention aims to remedy these drawbacks and to address this requirement through enabling collection under conditions compatible with routine laboratory examination of a large proportion of the cellular material, in particular RNA and DNA, of the cells concerned, in good condition.

To this end, a first aspect of the present invention provides a method for collecting cellular material of particular cells present in a liquid, characterized in that it includes:

a step of inserting the liquid into a compartment through an upper opening of the compartment, said compartment having a lower opening, a filter being positioned between the two openings the micropores of which have an intermediate diameter between that of said particular cells and that of other cells,

a filtration step during which most of the liquid and said other cells pass through the filter,

a step of lysis of the cells retained on the filter, and

a step of recovery of cellular material from the cells that have undergone lysis on the filter.

The method of the present invention enables cellular material to be collected quickly and efficiently.

As a general rule, the cellular material recovered using the present invention is the genetic material of the cells. The method of the present invention thus enables the recovery, directly on the filter and practically without losses, of the genetic material of scarce cells, up to a single isolated cell. Thus a high proportion of the genetic material is recovered from the cells concerned, in good condition, under conditions compatible with routine laboratory examination.

According to particular features, the method of the present invention, as succinctly stated hereinabove, includes, after the lysis step, a step of amplification of the DNA and/or the RNA and, during the recovery step, amplified genetic material is recovered from the cells that have undergone lysis.

According to particular features, during the amplification step, uniform amplification is effected preserving the quantitative aspect of the DNA and the RNA.

According to particular features, the method of the present invention as succinctly defined hereinabove further includes a detection step during which the amplified DNA is used as a matrix for detecting at least one mutation of the gene coding for sensitivity or resistance to at least one target therapy.

According to particular features, the method of the present invention as succinctly defined hereinabove further includes a detection step during which the amplified DNA is used as a matrix for detecting a variation of the level of expression of genes coding for sensitivity or resistance to the target therapies.

According to particular features, the method of the present invention as succinctly defined hereinabove further includes a detection step during which RNA is converted into cDNA and said cDNA is used to detect the level of expression of genes coding for sensitivity or resistance to the target therapies.

According to particular features, during the detection step, a quantitative polymerized chain reaction (PCR) is carried out in real time.

According to particular features, during the detection step, at least one forward and reverse primer pair is used to amplify a sequence of predetermined interest.

According to particular features, during the detection step, at least one pair of probes is used.

According to particular features, at least two probes of a pair of probes are coupled to two different fluorochromes and are defined so that one recognizes the mutated sequence and the other recognizes the normal sequence.

According to particular features, at least one pair of primers and one pair of probes are adapted to detect the G12D mutation of the K-ras gene coding for resistance to Erlotinib and to Gefitinib.

According to particular features, at least one primer includes at least 80% of the sequence AGGCCTGCTGAAAATGACTGAATAT.

According to particular features, at least one primer includes at least 80% of the sequence TCGTCCACAAAATGATTCTGAATTAGCT.

According to particular features, at least one probe includes at least 80% of the sequence TTGGAGCTGGTGGCGT.

According to particular features, at least one probe includes at least 80% of the sequence TGGAGCTGATGGCGT.

According to particular features, at least one pair of primers and one pair of probes are adapted to detect the G12V mutation of the K-ras gene coding for resistance to Erlotinib and to Gefitinib.

According to particular features, at least one primer includes at least 80% of the sequence AGGCCTGCTGAAAATGACTGAATAT.

According to particular features, at least one primer includes at least 80% of the sequence TCGTCCACAAAATGATTCTGAATTAGCT.

According to particular features, at least one probe includes at least 80% of the sequence TTGGAGCTGGTGGCGT.

According to particular features, at least one probe includes at least 80% of the sequence TTGGAGCTGTTGGCGT.

According to particular features, at least one pair of primers and one pair of probes are adapted to detect the G13C mutation of the K-ras gene coding for resistance to Erlotinib and to Gefitinib.

According to particular features, at least one primer includes at least 80% of the sequence AGGCCTGCTGAAAATGACTGAATAT.

According to particular features, at least one primer includes at least 80% of the sequence TCGTCCACAAAATGATTCTGAATTAGCT.

According to particular features, at least one probe includes at least 80% of the sequence TTGGAGCTGGTGGCGT.

According to particular features, at least one probe includes at least 80% of the sequence TTGGAGCTGGTTGCGT.

According to particular features, at least one pair of primers and one pair of probes are adapted to detect the L858R mutation of the EGFR gene coding for increased sensitivity to Erlotinib and to Gefitinib.

According to particular features, at least one primer includes at least 80% of the sequence GCAGCATGTCAAGATCACAGATTT.

According to particular features, at least one primer includes at least 80% of the sequence CCTCCTTCTGCATGGTATTCTTTCT.

According to particular features, at least one probe includes at least 80% of the sequence CAGTTTGGCCAGCCCA.

According to particular features, at least one probe includes at least 80% of the sequence CAGTTTGGCCCGCCCA.

According to particular features, the method as succinctly stated hereinabove includes, before the filtration step, a step of application of a formaldehyde-free fixing buffer to the liquid containing the particular cells.

This specifically hardens the particular cells searched for, without degrading the genetic material.

According to particular features, during the insertion step, the compartment has the general shape of a syringe in which the filter is positioned between the two openings.

Thanks to these features, a piston can be applied in the upper opening without modifying the position of the filter.

According to particular features, during the filtration step, suction is applied by pressure reduction below the filter. Thanks to these features, filtration is more efficient and faster than with no suction.

According to particular features, after the filtration step and before the recovery step, the compartment is placed over an Eppendorf tube. This achieves direct passage from the filter to an Eppendorf tube.

According to particular features, before the recovery step a piston including a central punch mobile inside the piston is positioned in the upper opening of the compartment.

According to particular features, the central mobile punch has a pointed lower end having a star shape.

According to particular features, the pointed lower end has a star shape.

According to particular features, during the recovery step, pressure is applied to the upper part of the punch to perforate the filter with the point of the punch.

According to particular features, the piston including means for immobilizing the punch longitudinally, during the recovery step, the punch is caused to pivot inside the piston to disengage it from said immobilizing means, before applying pressure to its upper part.

According to particular features, during the recovery step, vertical downward pressure is applied to the piston to cause the content of the compartment to pass into a tube placed below the compartment.

Thanks to each of these features, the cellular material of the particular cells can be extracted without damage through the perforation in the filter.

According to particular features, after the filtration step and before the recovery step, the content of the compartment is isolated by plugging the lower opening with a membrane.

According to particular features, said membrane is positioned under a strip surrounding the lower opening of each compartment. This provides a seal and retention of the lysis liquid above the filter.

According to particular features, said membrane is an adhesive membrane.

Thanks to each of these features, the content of the compartment is isolated from the environment between filtration and recovery of the cellular material of the particular cells.

According to particular features, during the recovery step, pressure is applied to the upper part of the punch to perforate the membrane isolating the content of the compartment.

According to particular features, the filter is produced in polycarbonate treated with a hydrophilic surface treatment. Using such a filter improves the retention of the particular cells and reduces the adhesion of the cellular material to be recovered.

According to particular features, the filter has a pore diameter centered on 7.5 μm. Thus, because of the dispersion of the diameters, practically none of the pores has a diameter greater than 8 μm.

Using this pore diameter, less than the diameter of the pores conventionally used for cytological analysis filters, enables them to be spaced farther apart, which reduces the number of contiguous pores and prevents the loss of the particular cells.

A second aspect of the present invention provides a device for collecting genetic material of particular cells present in a liquid, characterized in that it includes:

a compartment including an upper opening and a lower opening, a filter being positioned between the two openings the micropores of which have an intermediate diameter between that of said particular cells and that of other cells,

means for insertion of the liquid into said compartment through the upper opening,

filtration means causing most of the liquid and said other cells to pass through the filter,

means for lysis of the cells retained on the filter, and

means for recovery of cellular material of the cells that have undergone lysis on the filter.

A third aspect of the present invention provides a molecular biology kit including at least two primers and/or two probes from the following sequences:

AGGCCTGCTGAAAATGACTGAATAT,
TCGTCCACAAAATGATTCTGAATTAGCT,
TTGGAGCTGGTGGCGT,
TGGAGCTGATGGCGT,
AGGCCTGCTGAAAATGACTGAATAT,
TCGTCCACAAAATGATTCTGAATTAGCT,
TTGGAGCTGGTGGCGT,
TTGGAGCTGTTGGCGT,
AGGCCTGCTGAAAATGACTGAATAT,
TCGTCCACAAAATGATTCTGAATTAGCT,
TTGGAGCTGGTGGCGT,
TTGGAGCTGGTTGCGT,
GCAGCATGTCAAGATCACAGATTT,
CCTCCTTCTGCATGGTATTCTTTCT,
CAGTTTGGCCAGCCCA,
and
CAGTTTGGCCCGCCCA.

According to particular features, the kit of the present invention, as succinctly stated above, further includes a device of the present invention as succinctly defined hereinabove.

The advantages, objects and features of this device and this kit being similar to those of the method of the present invention, as succinctly stated hereinabove, they are not given here.

Other advantages, aims and features of the present invention will emerge from the following description given by way of nonlimiting explanation with reference to the appended drawings, in which:

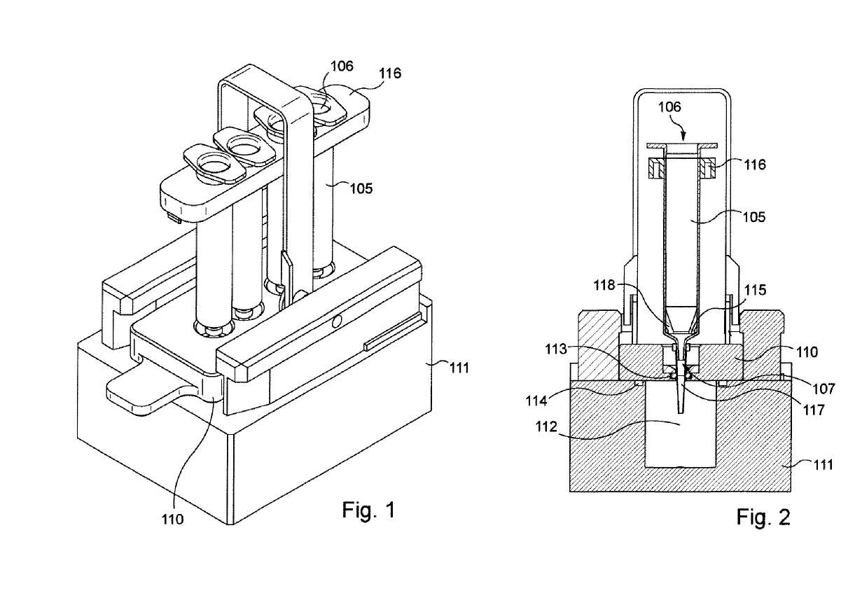

FIG. 1 represents diagrammatically in perspective parts of two particular embodiments of the device of the present invention used in a first phase of the method of the present invention,

FIG. 2 represents diagrammatically in section parts of two particular embodiments of the device of the present invention used in a first phase of the method of the present invention,

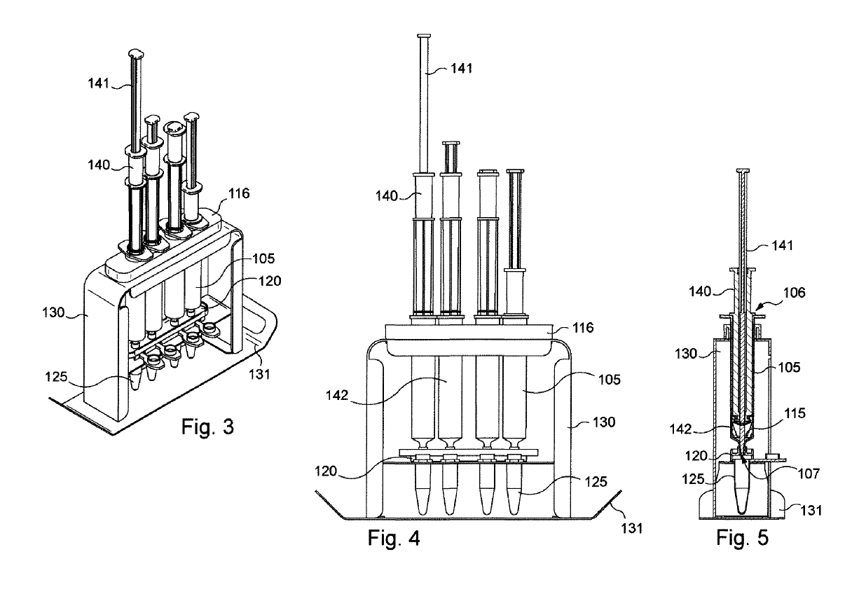

FIG. 3 represents diagrammatically in perspective parts of a first particular embodiment of the device of the present invention,

the FIG. 4 represents diagrammatically in elevation parts of the first particular embodiment of the device of the present invention,

FIG. 5 represents diagrammatically in section parts of the first particular embodiment of the device of the present invention,

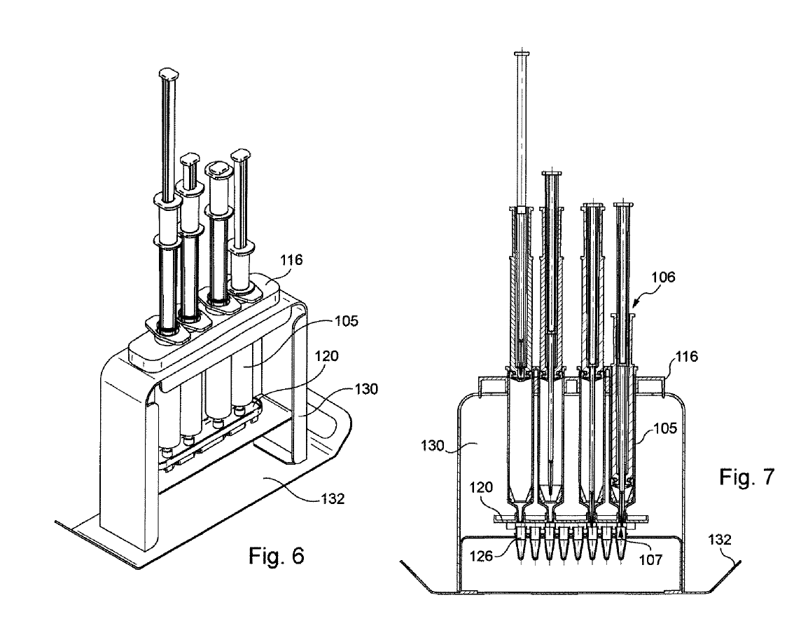

FIG. 6 represents diagrammatically in perspective parts of a second particular embodiment of the device of the present invention,

FIG. 7 represents diagrammatically in section parts of the second particular embodiment of the device of the present invention,

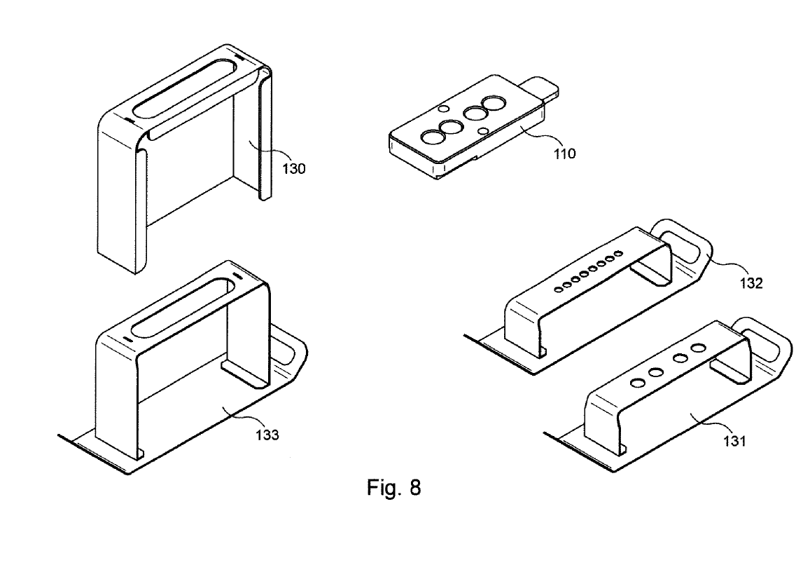

FIG. 8 represents diagrammatically in perspective parts of two particular embodiments of the device of the present invention used in a second phase of the method of the present invention, and

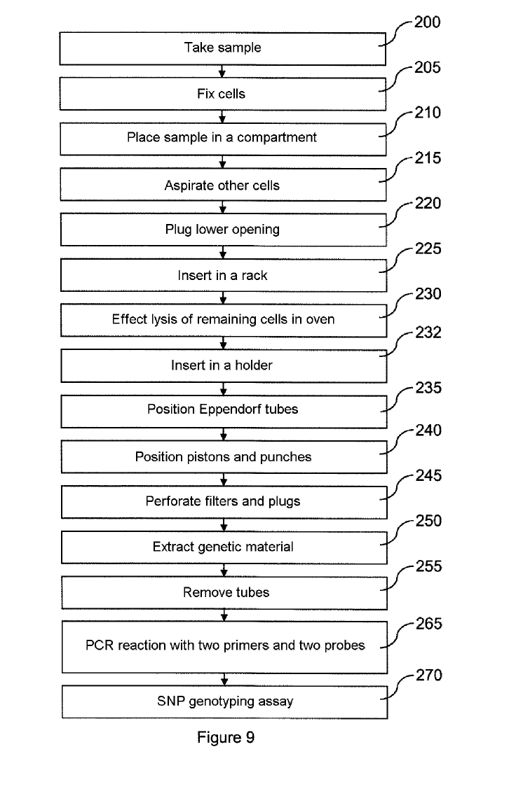

FIG. 9 represents in flowchart form the steps executed in one particular embodiment of the method of the present invention.

As seen in FIGS. 1 and 2, in the two embodiments shown in the figures, the device for collecting cellular, herein genetic, material includes a number of (here four) compartments 105 in the form of syringes each having an upper opening 106 and a lower opening 107. A washer or ring 118 placed in the lower opening 107 retains a filter 115. The filters 115 are microperforated and are stuck to the washers or rings 118 and then inserted at the distal end, i.e. at the bottom, of the four compartments 105 in the form of syringes. For example, the filter 115 is produced in polycarbonate with a hydrophilic surface treatment. The use of such a filter improves the rate of retention of the particular cells and reduces the adhesion of the cellular material to be recovered. The filter 115 has a pore diameter centered on 7.5 μm, for example. Because of the dispersion of the diameters, virtually none of the pores has a diameter exceeding 8 μm.

At the small opening, or lower opening, of each compartment 105 there is placed, in sealed manner, a tip 117 to prevent potential contamination of the end of the compartment 105 by splashes from the reservoir 112. These compartments 105 are assembled together by a plastic material strip 116 to constitute a single part. The combination produced in this way of the four compartments 105, the upper strip 116, the four pistons 140 and the plug strip 120 (described hereinafter) is disposable.

In the first phase of the method for collecting the cellular material, the compartments 105 are supported by a plate 110 inserted into a support 111 including a compartment connected to a reservoir 112 below the lower surface of the plate 110 via which aspiration can be effected. An O-ring 113 seals the connection between a tip 117 and the plate 110. An O-ring 114 seals the connection between the plate 110 and the support 111. The seal provided by the O-rings 113 and 114 enables aspiration of the content of the compartments.

During aspiration via the reservoir 112, some particular cells of the liquid present in the compartment 105, of greater diameter, are retained by the filter 115 whereas most of the liquid and the small cells are aspirated out of the compartment 105 through the filter 115.

At the end of the first phase of the method for recovering cellular material, the compartments 105 are removed from the part of the device shown in FIGS. 1 and 2 and the tips 117 are removed from the lower openings of the compartments 105. A plug strip 120 is then inserted into the tip of the compartments 105 in the form of syringes. This strip has four perforations which receive the tips at the lower ends of the compartments. A plastic film is stuck to the lower face of this plug strip 120 and constitutes a fluid-tight, preferably adhesive, membrane.

The content of the compartment 105 is thus isolated from the environment until the cellular material of the particular cells is recovered.

During the second phase of the method, the strip 116, carrying the compartments 105, associated with the plug strip 120, is placed on a plastic material rack 133. The cells present in the compartment 105 then undergo lysis in an oven. To this end, after the addition of reagents for the lysis of the cellular membranes of the cells searched for and uniform amplification of the DNA or RNA, the rack 133, keeping the compartments 105 vertical, is transported into an oven. On leaving the oven, after cooling, the Eppendorf tubes are placed on the lower support 131 of the holder 130. The strip 116 carrying the compartments 105 associated with the plug strip 120 is placed on the holder 130 and the holder 130 is placed on the lower support 131.

A piston 140 provided with a central axial punch 141 terminating in a point 142 is then inserted through the upper opening 106 of each compartment 105. In section, this point 142 preferably has a star shape, for example with four branches, the point then being cruciform.

An Eppendorf tube 125 held in position by a shelf-like portion of the holder 130 is placed below each compartment 105.

FIGS. 3 and 4 show the respective successive positions of the piston 140 and the punch 141 during the second phase of the method. On the left in each of these FIGS. 3 and 4, the piston 140 and the punch 141 are practically entirely outside the compartment 106 and the upper part of the punch 141 projects vertically above the piston 140.

Then, by application of a rotation, the punch is moved from a safe position in which the punch 141 cannot slide longitudinally inside a piston 140 because of a mechanical abutment, to an activation position in which the punch can slide longitudinally inside the piston 140. A downward vertical force is then applied to the top of the punch 141 causing the point 142 of this punch 141 to descend in the body of the compartment 106, toward the filter 115. As this descent continues, the point 142 first pierces the filter 115 and then the membrane 120 until it penetrates slightly into the tube 125. The punch 141 is then retained in position by contact with the lower opening of the compartment 105. Finally, as shown on the right in FIGS. 3 and 4, with the punch 141 retained in position, a downward force is applied to the piston 140 in order for it to push the content of the compartment 105 toward the tube 125, via the hole formed in the filter 115 and in the membrane 120 by the point of the punch 142.

The Eppendorf tubes 125 are then removed from the lower support 131 by withdrawing toward the top of the holder 130 the compartments 105 and the membrane 120, for analysis of the amplified genetic material, notably the DNA or the RNA of the cells of interest, collected in these tubes 125.

Note that the plate 110, the rack 133, the holder 130 and the support 131 are reusable.

In the second embodiment, shown in FIGS. 1, 2, 6 and 7, a support 131 of the holder 130 is replaced by a support 132 having eight openings instead of four. The four Eppendorf tubes 125 are replaced by a strip of eight Eppendorf tubes 126 of smaller capacity, typically 0.25 ml instead of 1.5 ml. These Eppendorf tubes are suited to another way of recovering cellular material enabling extraction of the amplified genetic material directly on the filter but in a smaller volume. Such a volume can then be contained in Eppendorf tubes directly suitable for a reverse transcription polymerase chain reaction (RT-PCR) in real time. Note that, when using eight Eppendorf tubes, four tubes are used to collect the amplified genetic material and four tubes are used to produce positive or negative controls.

As seen in FIG. 9, the method of the present invention includes first, in a manner known in the art, a step 200 of sampling a liquid to be analyzed, for example blood, which where appropriate is diluted or filtered. During a step 205, a formaldehyde-free fixing buffer is applied in order to fix, i.e. harden, specifically the particular cells searched for, without degrading their genetic material.

This fixing buffer consists, for example, of “PBS”, a buffer of phosphate, of saponin for lysis of the red globules of bovine serum albumin (BSA) for preserving the morphology of the cells of the calcium chelator ETDA, sodium hydroxide (NaOH) for adjusting the pH to 7.2, and RCL2, a cell fixative that does not degrade their genetic material. Note that formaldehyde is not used because it induces breaks in the genetic material.

During a step 210, the liquid resulting from the step 205 is placed in a compartment 105 that ends in a filter with micropores that have a diameter between that of the cells searched for and that of the other cells of the liquid sample. Alternatively, the fixing of the step 205 is effected inside the compartment 105, after the step 210.

During a step 215, suction is applied by pressure reduction under the filter. Most of the liquid and the cells of diameter smaller than that of the pores of the filter 115 then pass through the filter 115. On the other hand, the cells searched for of diameter less than that of the pores of the filter 115 are retained above the filter 115, in the compartment 105.

During a step 220, the compartments are removed from the plate 110, the tips 117 are removed from the compartments 105, and the remaining content in the compartment 105 is isolated by plugging the lower opening by a plug strip covered in its lower portion by an adhesive membrane 120.

During a step 225, the compartments 105 associated with the plug strip 120 and the strip 116 are inserted in the rack 133, the strip 116 being held by this rack 133.

During a step 230, the cells retained on the filter undergo lysis by known techniques. To this end, after addition of the reagents for the lysis of the cell membranes of the cells searched for and uniform amplification of the genetic material preserving the quantitative aspect, the rack 133, holding the compartments 105 vertical, is kept in an oven for a known period.

After removal from the oven, during a step 232, the strip 116, the compartments 105 and the plug strip 120 are removed from the rack 133 and placed on the holder 130.

During a step 235, each compartment 105 is placed over an Eppendorf tube 125 or 126, positioning this tube on the lower support 131 or 132, respectively, after which the holder 130 with the strip 116, the compartments 105 and the plug strip 120 is placed on the lower part 131 or 132, respectively.

During a step 240, a piston 140 provided with a central axial punch 141 mobile relative to the piston 140 and terminating at a point 142 inside the compartment 105 is inserted via the upper opening 106 of each compartment 105. In cross section, this point 142 preferably has a star shape, for example with four branches, the point then being cruciform.

During a step 245, the punch is turned to remove it from its safety position. Pressure is then applied to the upper part of the punch 141 to cause it to descend along the longitudinal axis of the compartment 105, guided by the piston 140. During this longitudinal movement, the point 142 of the punch 141 perforates successively the filter 115 and the plug or the adhesive membrane 120.

During a step 250, pressure is applied to the rest of the piston 140 so that the remaining content of the compartment 105 passes through the filter 115 and the plug or the membrane 120 and reaches the Eppendorf tube via the opening around the point 142 of the punch 141.

Thus the genetic material of the particular cells is extracted without damage through the perforation in the filter.

During a step 255, the support 131 or 132 and the Eppendorf tubes 125 or 126 are removed after removing the compartments 105 and the membrane 120 through the top of the holder 130.

During steps 265 and 270 an analysis is effected of the genetic material, notably the DNA or the RNA, of the cells searched for, collected in these tubes 125 or 126. The amplified DNA is used as a matrix for detecting mutations in sensitivity or resistance to the target therapies. Moreover, or alternatively, the complementary DNA (cDNA) produced from the RNA by RT conversion and then amplified is used as a matrix for detecting the level of expression of genes coding for sensitivity or resistance to the target therapies.

A defined volume of the amplified genetic material, notably DNA, is sampled to detect the mutations in sensitivity or resistance to the target therapies using forward and reverse primer pairs and pairs of probes during a quantitative polymerized chain reaction (PCR) in real time.

The principle of searching for mutations in sensitivity or resistance to the target therapies used in the embodiment shown is as follows. Information as to the presence or absence of a localized mutation at the level of a gene is provided by single nucleotide polymorphism (SNP) genotyping assay. The first step 265 of the SNP genotyping assay is a quantitative PCR reaction in real time using two primers to amplify the sequence of interest and two probes, for example of TaqMan (Registered Trade Mark) type. One of the probes recognizes the mutated sequence and the other recognizes the normal sequence. The two probes are associated with different fluorochromes, for example VIC for the probe hybridizing with the normal sequence and FAM for the probe hybridizing with the mutated sequence. The second step 270 uses an SNP genotyping assay program measuring the initial fluorescence and the final fluorescence emitted by the FAM and/or VIC fluorochromes. This program enables a distinction to be made between the various sequences present in each sample:

an increase in the fluorescence only in VIC indicates a homozygote profile for the normal sequence,

an increase in the fluorescence only in FAM indicates a homozygote profile for the mutated sequence,

an increase in the fluorescence both in VIC and in FAM indicates a heterozygote profile.

The following sequences of primers and probes (forward and reverse, 5′ to 3′) are used for the detection of mutations, for example:

For the G12D mutation of the K-ras gene coding for resistance to Erlotinib and to Gefitinib:

forward primer AGGCCTGCTGAAAATGACTGAATAT,
reverse primer TCGTCCACAAAATGATTCTGAATTAGCT,
“native” probes, color “VIC” TTGGAGCTGGTGGCGT,
“mutated” probe, color “FAM” TGGAGCTGATGGCGT.

For the G12V mutation (coding “12”) of the K-ras gene coding for resistance to Erlotinib and to Gefitinib:

forward primer AGGCCTGCTGAAAATGACTGAATAT,
reverse primer TCGTCCACAAAATGATTCTGAATTAGCT,
“native” probes, color “VIC” TTGGAGCTGTTGGCGT,
“mutated” probe, color “FAM” TTGGAGCTGTTGGCGT.

For the G13C mutation of the K-ras gene coding for resistance to Erlotinib and to Gefitinib:

forward primer AGGCCTGCTGAAAATGACTGAATAT,
reverse primer TCGTCCACAAAATGATTCTGAATTAGCT,
“native” probes, color “VIC” TTGGAGCTGGTTGCGT,
“mutated” probe, color “FAM” TTGGAGCTGGTTGCGT.

For the L858R mutation of the EGFR gene coding for increased sensitivity to Erlotinib and to Gefitinib:

forward primer GCAGCATGTCAAGATCACAGATTT,
reverse primer CCTCCTTCTGCATGGTATTCTTTCT,
“native” probes, color “VIC” CAGTTTGGCCCGCCCA,
“mutated” probe, color “FAM” CAGTTTGGCCCGCCCA.

The meaning of “forward”, “reverse”, and “5′ to 3′” are well known to the person skilled in the art. The probes match between the two primers and reveal the presence or the absence of a mutation by virtue of their associated fluorescence color. The color FAM is in the blue range of colors and the color VIC is in the green range of colors. Measuring the intensity of the fluorescence in each of these colors by the PCR apparatus discriminates normal genes, mutated homozygotes genes, and mutated heterozygote genes. Fifty amplification cycles are effected, for example.

In other embodiments, a defined volume of the genetic material, notably RNA converted into cDNA by RT (reverse transcription) and amplified is sampled to detect the level of expression of the gene coding for sensitivity or resistance to the target therapies using forward and reverse primer pairs and a probe and during a quantitative PCR (polymerase chain reaction) in real time, for example with 50 cycles.

As will be clear after reading the foregoing description, the method, the device and the kit of the present invention enable collection and amplification under conditions compatible with routine laboratory examination of a large proportion of the genetic material of the cells concerned, in good condition, even if the sample contains only one of the cells searched for.

1. Method for collecting cellular material of particular cells present in a liquid, characterized in that it includes:
a step (210) of inserting the liquid into a compartment (105) through an upper opening (106) of the compartment, said compartment having a lower opening (107), a filter (115) being positioned between the two openings the micropores of which have an intermediate diameter between that of said particular cells and that of other cells,
a filtration step (215) during which most of the liquid and said other cells pass through the filter,
a step (230) of lysis of the cells retained on the filter, and
a step (245, 250) of recovery of cellular material from the cells that have undergone lysis on the filter.

2. Method according to claim 1, characterized in that it includes, after the lysis step, a step of amplification of the DNA and/or the RNA and, during the recovery step, amplified genetic material is recovered from the cells that have undergone lysis.

3. Method according to claim 2, characterized in that, during the amplification step, uniform amplification is effected preserving the quantitative aspect of the DNA or the RNA.

4. Method according to claim 1, characterized in that it further includes a detection step (265, 270) during which the amplified DNA is used as a matrix for detecting at least one mutation of the gene coding for sensitivity or resistance to at least one target therapy.

5. Method according to claim 1, characterized in that it further includes a detection step (265, 270) during which the amplified DNA is used as a matrix for detecting a variation of the level of expression of genes coding for sensitivity or resistance to the target therapies.

6. Method according to claim 1, characterized in that it further includes a detection step (265, 270) during which RNA is converted into cDNA and said cDNA is used to detect the level of expression of genes coding for sensitivity or resistance to the target therapies.

7. Method according to claim 4, characterized in that, during the detection step (265, 270), a quantitative polymerized chain reaction (PCR) is carried out in real time.

8. Method according to claim 4, characterized in that, during the detection step (265, 270), at least one forward and reverse primer pair is used to amplify a sequence of predetermined interest.

9. Method according to claim 4, characterized in that, during the detection step (265, 270), at least one pair of probes is used.

10. Method according to claim 9, characterized in that at least two probes of a pair of probes are coupled to two different fluorochromes and are defined so that one recognizes a mutated sequence and the other recognizes a normal sequence.

11. Method according to claim 7, characterized in that at least one pair of primers and one pair of probes are adapted to detect of one of the following mutations:
the G12D mutation of the K-ras gene coding for resistance to Erlotinib and to Gefitinib,
the G12V mutation of the K-ras gene coding for resistance to Erlotinib and to Gefitinib,
the G13C mutation of the K-ras gene coding for resistance to Erlotinib and to Gefitinib, and
the L858R mutation of the EGFR gene coding for increased sensitivity to Erlotinib and to Gefitinib.

12. Method according to claim 11, characterized in that there are at least two primers and/or at least two probes from the following sequences:

| | |
|---|---|
| AGGCCTGCTGAAAATGACTGAATAT, | (SEQ ID NO: 1) |
| TCGTCCACAAAATGATTCTGAATTAGCT, | (SEQ ID NO: 2) |
| TTGGAGCTGGTGGCGT, | (SEQ ID NO: 3) |
| TGGAGCTGATGGCGT, | (SEQ ID NO: 4) |
| AGGCCTGCTGAAAATGACTGAATAT, | (SEQ ID NO: 5) |
| TCGTCCACAAAATGATTCTGAATTAGCT, | (SEQ ID NO: 6) |
| TTGGAGCTGGTGGCGT, | (SEQ ID NO: 7) |
| TTGGAGCTGTTGGCGT, | (SEQ ID NO: 8) |
| AGGCCTGCTGAAAATGACTGAATAT, | (SEQ ID NO: 9) |
| TCGTCCACAAAATGATTCTGAATTAGCT, | (SEQ ID NO: 10) |
| TTGGAGCTGGTGGCGT, | (SEQ ID NO: 11) |
| TTGGAGCTGGTTGCGT, | (SEQ ID NO: 12) |
| GCAGCATGTCAAGATCACAGATTT, | (SEQ ID NO: 13) |
| CCTCCTTCTGCATGGTATTCTTTCT, | (SEQ ID NO: 14) |
| CAGTTTGGCCAGCCCA | (SEQ ID NO: 15) |
| and | |
| CAGTTTGGCCCGCCCA. | (SEQ ID NO: 16) |

13. Method according to claim 1, characterized in that, before the recovery step (245, 250) a piston (140) including a central punch (141) mobile inside the piston is positioned in the upper opening (106) of the compartment (105).

14. Method according to claim 13, characterized in that the central mobile punch (141) has a pointed lower end (142) having a star shape.

15. Method according to claim 1, characterized in that after the filtration step (215) and before the recovery step (245, 250) the content of the compartment (105) is isolated by plugging the lower opening (107) with a membrane positioned under a strip (120) surrounding the lower opening (107) of each compartment (105).

16. Method according to claim 1, characterized in that the filter (115) is produced in polycarbonate treated with a hydrophilic surface treatment.

17. Method according to claim 1, characterized in that the filter (115) has a pore diameter centered on 7.5 μm.

18. Device for collecting genetic material of particular cells present in a liquid, characterized in that it includes:
a compartment (105) including an upper opening (106) and a lower opening (107), a filter (115) being positioned between the two openings the micropores of which have an intermediate diameter between that of said particular cells and that of other cells,
means for insertion of the liquid into said compartment through the upper opening,
filtration means (111, 112) causing most of the liquid and said other cells to pass through the filter,
means for lysis of the cells retained on the filter, and
means for recovery of cellular material of the cells that have undergone lysis on the filter.

19. Molecular biology kit for use in a device according to claim 18, characterized in that it includes at least two primers and/or two probes from the following sequences:

| | |
|---|---|
| AGGCCTGCTGAAAATGACTGAATAT, | (SEQ ID NO: 1) |
| TCGTCCACAAAATGATTCTGAATTAGCT, | (SEQ ID NO: 2) |
| TTGGAGCTGGTGGCGT, | (SEQ ID NO: 3) |
| TGGAGCTGATGGCGT, | (SEQ ID NO: 4) |
| AGGCCTGCTGAAAATGACTGAATAT, | (SEQ ID NO: 5) |
| TCGTCCACAAAATGATTCTGAATTAGCT, | (SEQ ID NO: 6) |
| TTGGAGCTGGTGGCGT, | (SEQ ID NO: 7) |
| TTGGAGCTGTTGGCGT, | (SEQ ID NO: 8) |
| AGGCCTGCTGAAAATGACTGAATAT, | (SEQ ID NO: 9) |
| TCGTCCACAAAATGATTCTGAATTAGCT, | (SEQ ID NO: 10) |
| TTGGAGCTGGTGGCGT, | (SEQ ID NO: 11) |
| TTGGAGCTGGTTGCGT, | (SEQ ID NO: 12) |
| GCAGCATGTCAAGATCACAGATTT, | (SEQ ID NO: 13) |
| CCTCCTTCTGCATGGTATTCTTTCT, | (SEQ ID NO: 14) |
| CAGTTTGGCCAGCCCA, | (SEQ ID NO: 15) |
| and | |
| CAGTTTGGCCCGCCCA. | (SEQ ID NO: 16) |

20. Kit according to claim 19, characterized in that it further includes a device for collecting genetic material of particular cells present in a liquid, the device comprising:
a compartment (105) including an upper opening (106) and a lower opening (107), a filter (115) being positioned between the two openings the micropores of which have an intermediate diameter between that of said particular cells and that of other cells,
means for insertion of the liquid into said compartment through the upper opening,
filtration means (111, 112) causing most of the liquid and said other cells to pass through the filter,
means for lysis of the cells retained on the filter, and
means for recovery of cellular material of the cells that have undergone lysis on the filter.


SEQUENCE LISTING (16 sequences)

SEQ ID NO: 1 - DNA, 25 bp, Artificial Sequence
  Synthetic Primer
aggcctgctgaaaatgactgaatat

SEQ ID NO: 2 - DNA, 28 bp, Artificial Sequence
  Synthetic Primer
tcgtccacaaaatgattctgaattagct

SEQ ID NO: 3 - DNA, 16 bp, Artificial Sequence
  Synthetic Primer
ttggagctggtggcgt

SEQ ID NO: 4 - DNA, 15 bp, Artificial Sequence
  Synthetic Primer
tggagctgatggcgt

SEQ ID NO: 5 - DNA, 25 bp, Artificial Sequence
  Synthetic Primer
aggcctgctgaaaatgactgaatat

SEQ ID NO: 6 - DNA, 28 bp, Artificial Sequence
  Synthetic Primer
tcgtccacaaaatgattctgaattagct

SEQ ID NO: 7 - DNA, 16 bp, Artificial Sequence
  Synthetic Primer
ttggagctggtggcgt

SEQ ID NO: 8 - DNA, 16 bp, Artificial Sequence
  Synthetic Primer
ttggagctgttggcgt

SEQ ID NO: 9 - DNA, 25 bp, Artificial Sequence
  Synthetic Primer
aggcctgctgaaaatgactgaatat

SEQ ID NO: 10 - DNA, 28 bp, Artificial Sequence
  Synthetic Primer
tcgtccacaaaatgattctgaattagct

SEQ ID NO: 11 - DNA, 16 bp, Artificial Sequence
  Synthetic Primer
ttggagctggtggcgt

SEQ ID NO: 12 - DNA, 16 bp, Artificial Sequence
  Synthetic Primer
ttggagctggttgcgt

SEQ ID NO: 13 - DNA, 24 bp, Artificial Sequence
  Synthetic Primer
gcagcatgtcaagatcacagattt

SEQ ID NO: 14 - DNA, 25 bp, Artificial Sequence
  Synthetic Primer
cctccttctgcatggtattctttct

SEQ ID NO: 15 - DNA, 16 bp, Artificial Sequence
  Synthetic Primer
cagtttggccagccca

SEQ ID NO: 16 - DNA, 16 bp, Artificial Sequence
  Synthetic Primer
cagtttggcccgccca